(19) United States
(12) Patent Application Publication
(10) Pub. No.: US 2020/0215024 A1
(43) Pub. Date: Jul. 9, 2020

(54) CANNABINOID FORMULATIONS FOR TREATING ALCOHOL HANGOVER

(71) Applicant: Replennabis LLC, Westport, CT (US)

(72) Inventors: Kayla Berman, Tel Aviv (IL); Andra Weissberger, Santa Clarita, CA (US); Abraham J. Domb, Jerusalem (IL)

(21) Appl. No.: 16/737,413

(22) Filed: Jan. 8, 2020

Related U.S. Application Data
(60) Provisional application No. 62/789,838, filed on Jan. 8, 2019.

(51) Int. Cl. A61K 31/352; A61K 9/48; A61K 9/00; A61K 9/20; A61K 9/19; A61K 47/38; A61K 47/10; A61K 47/44; A61K 47/14; A61K 36/16; A61K 36/9068; A61K 36/33; A61K 36/258; A61K 36/288; A61K 36/82; A61K 36/8998; A61K 31/05; A61K 31/375; A61K 36/287

(52) U.S. Cl. CPC A61K 31/352; A61K 36/287; A61K 9/006; A61K 9/20; A61K 9/19; A61K 47/38; A61K 47/10; A61K 47/44; A61K 47/14; A61K 36/16; A61K 36/9068; A61K 36/33; A61K 36/258; A61K 36/288; A61K 36/82; A61K 36/8998; A61K 31/05; A61K 31/375; A61K 9/4825

(57) ABSTRACT

Compositions for reducing alcohol consumption and/or treating alcohol hangover contain a first active agent containing at least one cannabinoid compound, a second active agent effective in reducing one or more symptoms of alcohol hangover, and a carrier. In some forms, the carrier is an anhydrous liposphere concentrate (ALC) containing a lipid system including lipid, optionally a water miscible solvent, and one or more surfactants and/or one or more phospholipids. In a preferred embodiment, the carrier spontaneously forms a nanodispersion of nanolipids or lipospheres, upon addition to an aqueous medium, which contain the cannabinoid compound and other compounds from the active agents. The compositions can be used to form oral dosage formulations for delivery of the cannabinoid compound.

### CROSS-REFERENCE TO RELATED APPLICATIONS

This application claims the benefit of and priority to U.S. Provisional Application No. 62/789,838 filed Jan. 8, 2019, which is hereby incorporated by reference in its entirety.

### FIELD OF THE INVENTION

The invention is generally directed to controlling alcohol consumption and/or treating alcohol hangover.

### BACKGROUND OF THE INVENTION

Alcohol-induced hangover, defined by a series of symptoms, is the most commonly reported consequence of excessive alcohol consumption. Alcohol hangover develops when an elevated blood alcohol concentration falls considerably and peaks when it reaches almost zero. Hangover symptoms, which include headache, tiredness, concentration problems, thirst, dizziness, nausea, cognitive impairment, and mood changes may last for 24 hours or longer. Alcohol hangover may cause health problems, memory and learning impairment, workplace absenteeism, impaired job performance, reduced productivity, and poor academic achievement. Alcohol hangover is not simply the equivalent of dehydration. Other mechanisms, such as activation of the immune system, may play a role in the genesis of alcohol hangover. Up to now, most potential hangover cures have shown no effectiveness, whereas other cures reduce only some of the symptoms of alcohol hangover. Moreover, side effects such as neuritis, nausea, and central nervous system-related symptoms are frequently associated with hangover treatments.

There is a need for developing a treatment or cure that reduces alcohol consumption and/or alleviate symptoms of alcohol hangover.

There is also a need for developing oral formulations to deliver cannabinoids for the purpose of controlling alcohol consumption and/or treating alcohol hangover.

Therefore, it is the object of the present invention to provide cannabinoid-containing compositions for reducing alcohol consumption and/or treating alcohol hangover.

It is another object of the present invention to provide oral dosage formulations of such compositions to deliver cannabinoids.

It is another object of the present invention to provide methods of controlling alcohol consumption and/or treating alcohol hangover, using the cannabinoid-containing compositions and formulations.

### SUMMARY OF THE INVENTION

Compositions for reducing alcohol consumption and/or treating alcohol hangover contain a first active agent containing at least one cannabinoid compound, optionally a second active agent effective in reducing one or more symptoms of alcohol hangover, and a carrier or vehicle.

The first active agent may be a cannabinoid extract from one or more cannabis plants, an isolated cannabinoid compound of plant or animal origin, or a synthesized cannabinoid compound. Preferably, the cannabinoid compound is cannabidiol (CBD), tetrahydrocannabinol, or cannabinol. Most preferably, the cannabinoid compound is CBD.

The second active agent is effective in treating one or more symptoms of hangover such as headache, nausea or dizziness. In some embodiments, the second active agent is an herbal ingredient, such as an herb, an herbal extract or isolated herbal compound.

In some forms, the carrier/vehicle is an anhydrous liposphere concentrate (ALC) containing a lipid system including lipid, optionally a water miscible solvent, one or more surfactants and/or one or more phospholipids. In a preferred embodiment, the carrier spontaneously forms nanoparticles or lipospheres, upon addition to an aqueous medium, which entrap the cannabinoid compound and other compounds. The nanoparticles have an average diameter of between 0.01 and 100 μm, preferably less than 500 nm, most preferably less than 100 nm. The lipid from the lipid system forms the lipid core of the lipospheres, and the surfactants and phospholipids form the outer layer(s) of the lipospheres.

Useful lipids include monoglycerides, diglycerides, triglycerides, fatty acids, and combinations thereof. Preferably, the lipid system contains one or more tryglycerides. The water-miscible solvent may be ethanol, N-methylpyrrolidone, ethyl lactate, ethyl acetate, polyethylene glycol, glycerol, propylene glycol, or combinations thereof. Preferably, the water-miscible solvent is ethanol. The surfactants are preferably non-ionic surfactants, for example, TWEEN® surfactants (polysorbates), such as TWEEN® 20 (polysorbate 20), TWEEN® 65 (polysorbate 65), and TWEEN® 80 (polysorbate 80), SPAN® surfactants (sorbitan esters), such as SPAN® 80 (sorbitan oleate), BRIJ® surfactants (polyoxyethylene alkyl ethers), polyoxyethylene fatty acid ester surfactants such as CREMOPHOR® RH 40 (polyoxyl 40 hydrogenated castor oil), sodium dodecyl sulfate; and combinations thereof. Preferably, the surfactants are or contain TWEEN® 20, SPAN® 80, and CREMOPHOR® RH 40. In some embodiments, the carrier/vehicle includes at least a lipid system, one or more surfactants, and lecithin (as a carrier of phospholipids).

The compositions are formed into oral dosage units. In some embodiments, the compositions are loaded into capsules such as soft gelatin capsules. In some embodiments, the compositions are dispersed in an aqueous medium to form the oral dosage formulation. In some embodiments, the compositions are absorbed on an absorbent to form semi-dry or dry formulations, which can be further loaded into capsules such as gelatin capsules or compressed into tablets. In some embodiments, the compositions are added to an aqueous medium and further lyophilized to form a solid formulation. The solid formulation can be loaded in capsules such as gelatin capsules or compressed into tablets.

Alternatively, the carrier/vehicle of the compositions is a fast-dissolving film (FDF) containing one or more film-forming agents and optionally one or more surfactants. Preferably, the FDF dissolves within 10 minutes, five minutes, two minutes, or one minute in the oral cavity after contact with saliva. In some embodiments, the first active agent and optionally the second active agent are directly loaded into the FDF such that they can be released and/or absorbed in the oral cavity. In another embodiment, the first active agent and optionally the second active agent are first loaded into an ALC as described herein, which is then loaded into the FDF.

The second active agent can be formulated together with the first active agent in the same composition. Alternatively, the second active agent can be formulated in a separate composition, which can be the same or different from the design of the composition containing the first active agent. The second active agent can be administered before, after, or concurrently with the first active agent.

The compositions and formulations can be used to help control alcohol consumption and/or treat alcohol hangover when orally administered to a subject in need thereof any one of the compositions or formulations, before, during, or after alcohol consumption.

### DETAILED DESCRIPTION OF THE INVENTION

### I. Definitions

Use of the term “about” is intended to describe values either above or below the stated value in a range of approx. +/−10%; in other embodiments the values may range in value either above or below the stated value in a range of approx. +/−5.

As used herein, the term “derivative” refers to a compound with a structure similar to that of another (reference compound) but differing from it in respect to a particular component, functional group, atom, etc. The term “derivative” also refers to a compound which is formed from a parent compound by chemical reaction(s). The differences between suitable derivatives and their reference or parent compounds include, but are not limited to, replacement of one or more chemical groups with one or more different chemical groups, introducing one or more substituents to any hydrogen atoms, or reacting one or more chemical groups to introduce one or more substituents.

“Excipient” refers to all pharmaceutically inert components present in a composition or formulation other than the active ingredient or ingredients. They may include but are not limited to diluents, binders, lubricants, disintegrators, fillers, plasticizers, pigments, colorants, stabilizing agents, flavorings, conservatives, and glidants.

“Cannabinoid” refers to a class of diverse chemical compounds that acts on cannabinoid receptors in cells that alter neurotransmitter release in the brain. Cannabinoid compounds include endocannabinoids (produced naturally in the body by animals), phytocannabinoids (found in cannabis and some other plants), and synthetic cannabinoids (manufactured artificially).

“Herbal ingredient” refers to substance produced by, or derived, or isolated from herbs. In some forms, it can be a dried herb optionally in the form of a dried herb powder. In some forms, it can be an herb extract. In some forms, it can be a herbal compound isolated from herbs or chemically synthesized.

“Essential oil” is a concentrated hydrophobic liquid containing volatile (easily evaporated at room temperatures) chemical compounds from plants. Essential oils are also known as volatile oils, ethereal oils, aetherolea, or simply as the oil of the plant from which they were extracted, such as oil of clove. An essential oil is “essential” in the sense that it contains the “essence of” the plant's fragrance—the characteristic fragrance of the plant from which it is derived. The term “essential” used here does not mean indispensable, as with the terms “essential amino acid” or “essential fatty acid,” which are so called because they are nutritionally required by a given living organism.

“Liposphere” or “nano liposphere” refers to lipid-based water dispersible particles, composed of a hydrophobic lipid core, which is stabilized by a layer of amphiphilic molecules such as surfactants and/or phospholipids, as external coating. Lipospheres may have a particle size between 0.01 and 100 μm, preferably less than 500 nm, in diameter. Lipospheres can entrap agents or compounds dissolved or dispersed in the lipid core.

“Dispersible concentrate” or “pre-concentrate” refers to compositions which spontaneously form a nano-particulate dispersion in an aqueous medium, for example, in water upon dilution or in the gastric fluids after oral administration.

“Aqueous medium” refers to a water-based medium, i.e., a liquid medium in which water is the major component.

“Surfactant” refers to amphiphilic compounds generally recognized in the art as having surface active qualities. Surfactants generally include anionic, cationic, nonionic, and zwitterionic compounds.

The term “substituent” as used herein, include, but are not limited to: a halogen atom, an alkyl group, a heteroalkyl group, an alkenyl group, a heteroalkenyl group, an alkynyl group, a heteroalkynyl group, an aryl group, a heteroaryl group, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHCl₂, —CH₂NH₂, —NHCOH, —CHO, —COCl, —COF, —COBr, —COOH, —SO₃H, —CH₂SO₂CH₃, —PO₃H₂, —OPO₃H₂, —P(═O)(OR^{G1′})(OR^{G2′}), —OP(═O)(OR^{G1′})(OR^{G2′}), —BR^{G1′}(OR^{G2′}), —B(OR^{G1′})(OR^{G2′}), or -G′R^{G1′} in which -G′ is —O—, —S—, —NR^{G2′}—, —C(═O)—, —S(═O)—, —SO₂—, —C(═O)O—, —C(═O)NR^{G2′}-, —OC(═O)—, —NR^{G2′}C(═O)—, —OC(═O)O—, —OC(═O)NR^{G2′}—, —NR^{G2′}C(═O)O—, —NR^{G2′}C(═O)NR^{G3′}—, —C(═S)—, —C(═S)S—, —SC(═S)—, —SC(═S)S—, —C(═NR^{G2}′)—, —C(═NR^{G2′})O—, —C(═NR^{G2′})NR^{G3′}—, —OC(═NR^{G2′})—, —NR^{G2′}C(═NR^{G3′})—, —NR—, —C(═NR^{G2′})NR^{G3′}—, —OC(═NR^{G2′})—, —NR^{G2}′C(═NR^{G3}′)—, —NR^{G2′}SO₂—, —NR^{G2′}SO2NR^{G3′}—, —NR^{G2′}C(═S)—, —SC(═S)NR^{G2′}—, —NR^{G2′}C(═S)S—, —NR^{G2′}C(═S)NR^{G3′}—, —SC(═NR^{G2′})—, —C(═S)NR^{G2′}—, —OC(═S)NR^{G2′}—, —NR^{G2′}C(═S)O—, —SC(=O)NR^{G2′}—, —NR^{G2′}C(═O)S—, —C(═O)S—, —SC(═O)—, —SC(═O)S—, —C(═S)O—, —OC(═S)—, —OC(═S)O—, —SO₂NR^{G2′}—, —BR^{G2′}—, or —PR^{G2′}—,

wherein each occurrence of R^{G1′}, R^{G2′}, and R^{G3′} is, independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

It is understood that “substituent” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

The term “alkyl” refers to univalent groups derived from alkanes by removal of a hydrogen atom from any carbon atom. Alkanes represent saturated hydrocarbons, including those that are cyclic (either monocyclic or polycyclic). Alkyl groups can be linear, branched, or cyclic. Suitable alkyl groups can have one to 30 carbon atoms, i.e., C₁-C₃₀ alkyl. If the alkyl is branched, it is understood that at least four carbons are present. If the alkyl is cyclic, it is understood that at least three carbons are present.

The term “heteroalkyl” refers to alkyl groups where one or more carbon atoms are replaced with a heteroatom, such as, O, N, or S. Heteroalkyl groups can be linear, branched, or cyclic (either monocyclic or polycyclic). Suitable heteroalkyl groups can have one to 30 carbon atoms, i.e., C₁-C₃₀ heteroalkyl. If the heteroalkyl is branched, it is understood that at least three carbons and at least one heteroatom are present. If the heteroalkyl is cyclic, it is understood that at least two carbons and at least one heteroatom are present. The term “alkenyl” refers to univalent groups derived from alkenes by removal of a hydrogen atom from any carbon atom. Alkenes are unsaturated hydrocarbons that contain at least one carbon-carbon double bond. Alkenyl groups can be linear, branched, or cyclic (either monocyclic or polycyclic). Suitable alkenyl groups can have two to 30 carbon atoms, i.e., C₂-C₃₀ alkenyl. If the alkenyl is branched, it is understood that at least four carbons are present. If the alkenyl is cyclic, it is understood that at least three carbons are present.

The term “heteroalkenyl” refers to alkenyl groups in which one or more carbon atoms are replaced by a heteroatom. Heteroalkenyl groups can be linear, branched, or cyclic (either monocyclic or polycyclic). Suitable heteroalkenyl groups can have one to 30 carbon atoms, i.e., C₁-C₃₀ heteroalkenyl. If the heteroalkenyl is branched, it is understood that at least three carbons and at least one heteroatom are present. If the heteroalkenyl is cyclic, it is understood that at least two carbons and at least one heteroatom are present.

The term “alkynyl” refers to univalent groups derived from alkynes by removal of a hydrogen atom from any carbon atom. Alkynes are unsaturated hydrocarbons that contain at least one carbon-carbon triple bond. Alkynyl groups can be linear, branched, or cyclic (either monocyclic or polycyclic). Suitable alkynyl groups can have two to 30 carbon atoms, i.e., C₂-C₃₀ alkynyl.

If the alkynyl is branched, it is understood that at least four carbons are present. If alkynyl is cyclic, it is understood that at least five carbons are present.

The term “heteroalkynyl” refers to alkynyl groups in which one or more carbon atoms are replaced by a heteroatom. Heteroalkynyl groups can be linear, branched, or cyclic (either monocyclic or polycyclic). Suitable heteroalkynyl groups can have one to 30 carbon atoms, i.e., C₁-C30 heteroalkynyl. If the heteroalkynyl is branched, it is understood that at least three carbons and at least one heteroatom are present. If the heteroalkynyl is cyclic, it is understood that at least three carbons and at least one heteroatom are present. The term “aryl” refers to univalent groups derived from arenes by removal of a hydrogen atom from a ring atom. Arenes are monocyclic or polycyclic aromatic hydrocarbons. In polycyclic arenes, the rings can be attached together in a pendant manner or can be fused. Accordingly, in polycyclic aryl groups, the rings can be attached together in a pendant manner or can be fused. Suitable aryl groups can have six to 50 carbon atoms, i.e., C₆-C₅₀ aryl.

The term “heteroaryl” refers to univalent groups derived from heteroarenes by removal of a hydrogen atom from a ring atom. Heteroarenes are heterocyclic compounds derived from arenes by replacement of one or more methine (—C═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Hüickel rule (4n+2). Heteroarenes can be monocyclic or polycyclic. In polycyclic heteroarenes, the rings can be attached together in a pendant manner or can be fused. Accordingly, in polycyclic heteroaryl groups, the rings can be attached together in a pendant manner or can be fused. Suitable heteroaryl groups can have three to 50 carbon atoms, i.e., C₃-C₅₀ heteroaryl.

The term “room temperature” refers to a temperature between 20-25° C., such as about 20° C., about 21° C., about 22° C., about 23° C., about 24° C., or about 25° C.

The terms “treatment” and “treating” refer to the medical management of a subject with the intent to cure, ameliorate, stabilize, or prevent one or more symptoms of a disease, pathological condition, or disorder. This term includes active treatment toward the improvement of the disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms.

The term “preventing” refers to administering a pharmaceutical composition prior to the onset or exacerbation of one or more clinical symptoms or of a disease, pathological condition, or disorder to prevent a physical manifestation of aberrations associated with the disease, pathological condition, or disorder.

The term “effective amount” of a composition (e.g., a compound, com formulation) refers to a nontoxic but sufficient amount of the composition to provide the desired result. The exact amount required will vary depending on the severity of alcohol hangover.

### II. Cannabinoid Compositions

The clinical use of cannabinoids is limited by poor oral bioavailability, given that the oral route of administration is the most convenient route for patients. THC and CBD suffer from extremely low oral bioavailability (less than 8%) stemming from their lipophilic characteristics, poor solubility, and significant first pass metabolism (Zgair, et al., Am J Transl Res, 2016, 8(8):3448-59; Pertwee, Br J Pharmacol, 2008, 153:199-215; Duran-Lobato, et al., Drug Dev Ind Pharm, 2016, 42:190-8; and Grotenhermen, Clin Pharmacokinet, 2003, 42:327-60). These poorly water soluble and highly metabolized compounds fall into Class II category of drugs according to both the Biopharmaceutical Classification System (BCS) designed by Amidon et al. (Amidon, et al., Pharm Res, 1995, 12(3):413-20) and the Biopharmaceutical Drug Disposition Classification System (BDDCS) outlined by Wu and Benet (Wu and Benet, Pharm Res, 2005, 22(1):11-23).

Anhydrous liposphere compositions for reducing alcohol consumption and/or treating alcohol hangover having enhanced oral uptake contain a first active agent containing at least one cannabinoid compounds, optionally a second active agent effective in reducing alcohol hangover symptoms, and a carrier containing a lipid system, optionally a water-miscible solvent, and one or more surfactants and/or one or more phospholipids. The lipid system contains at least one lipid. The lipid component can be between 40 to about 90% of the total formulation that make the formulation. The other components of the formulation, surfactants between 1-20%, solvent 0-50% and the active agent can be from 1 to 50% of the formulation.

The anhydrous liposphere compositions can spontaneously form nanoparticles or nanodroplets which encapsulate the active agents, when reaching the aqueous phase of the gastrointestinal tract after oral administration. These compositions can enhance absorption of the active agents, especially cannabinoids, face when administered orally, through the following mechanisms: (1) enhanced dispensability in the aqueous milieu of the gastrointestinal tract; (2) increased intestinal surface area available for absorption via nanoparticles, preferably those with a size below 100 nm; (3) decreased intestinal first pass metabolism due to the use of lipid nanoparticles and surfactants; (4) enhanced lymphatic absorption which leads to bypass of the remaining metabolism occurring in the liver and a flatter absorption profile compared to direct oral administration.

Fast dissolving films (FDFs) for reducing alcohol consumption and/or treating alcohol hangover contain a first active agent containing at least one cannabinoid compounds, optionally a second active agent effective in reducing alcohol hangover symptoms, one or more film-forming agents, and, optionally, one or more surfactants. In some embodiments, the first active agent and optionally the second active agent are directly loaded into the FDF such that they can be released and/or absorbed in the oral cavity. In other embodiment, the first active agent and optionally the second active agent are first loaded into an ALC as described herein, which is then loaded into the FDF.

Preferably, the FDF dissolves within 10 minutes, five minutes, two minutes, or one minute in oral cavity after contact with saliva.

A. Therapeutic or Prophylactic Agents
1. First Active Agent

The cannabis plant has been at the center stage of modern medicine due to its pharmacological effects. Medical marijuana is approved in many countries for many medical conditions such as cachexia, cancer, chronic pain, epilepsy and disorders characterized by seizures, glaucoma, HIV, AIDS, multiple sclerosis, muscle spasticity and nausea. Progress has been made on several fronts in the use of cannabinoids for medical use. The major cannabinoids under research, clinically and pre-clinically, are Δ⁹-tetrahydrocannabinol (THC) and cannabidiol (CBD). While THC is considered as a psycoactive substance, CBD is often referred to as a non-psychoactive compound. Notably, cannabinoids such as CBD can reduce gene expression of tyrosine hydroxylase in the ventral tegmental area, and therefore decrease behavioral reinforcement associated with alcohol consumption (Viudez-Martínez, et al., Addict Biol, 2018, 23(1):154-164).

Combinations of drugs is a common clinical approach used to improve the clinical response to pharmacological treatment. Using natural herbal active agents together with cannabinoids may control alcohol addiction and relieve symptoms of alcohol hangover. For example, the combination of low doses of CBD plus naltrexone were more effective than either CBD or naltrexone alone in reducing alcohol consumption and the motivation to drink (Viudez-Martinez, et al., Br J Pharmacol, 2018, 175(16):3369-3378, doi: 10.1111/bph.14380).

The first active agent contains at least one cannabinoid compound such as THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), CBT (cannabicitran), and derivatives thereof. Additional cannabinoid compounds are disclosed in Aizpurua-Olaizola, et al., Journal of Natural Products, 2016, 79(2):324-31. Preferred cannabinoid compounds include cannabidiol (CBD), tetrahydrocannabinol (THC), and cannabinol (CBN).

Cannabinoids such as CBD can reduce gene expression of tyrosine hydroxylase in the ventral tegmental area. They can decrease behavioral reinforcement associated with alcohol consumption, thereby reducing alcohol consumption.

The first active agent may be a cannabinoid extract from one or more cannabis plants, an isolated cannabinoid compound from a plant origin (i.e., phytocannabinoids) or an animal origin (i.e., endocannabinoids), or a synthesized cannabinoid compound.

The cannabinoid extract may be obtained from commercial sources that extract the active agents using organic solvents such as alcohols or liquid carbon dioxide. Methods for effective extraction and isolation cannabinoid actives have been described in numerous publications such as in Leiman, et al., Talanta, 2018, 190:321-326; Fairbairn and Liebmann, J. Pharm. Pharmac., 1973, 25:150-155.

In a preferred embodiment, the first active agent is or contains CBD. CBD has several beneficial pharmacological effects. It is antipsychotic, neuroprotective, antiemetic and anti-inflammatory. Additionally, CBD can reduce and prevent symptoms of hangover, such as anxiety, depression, fatigue, insomnia, headaches, etc. CBD is considered to be safe and non-psychoactive; after using it, the physiological parameters like blood pressure, heart rate, and body temperature, are not altered.
2. Second Active Agent

The second active agent is effective in reducing alcohol hangover symptoms such as pain, headache, nausea and dehydration, eliminating toxins, reliving fogginess, and/or providing liver and metabolism support.

Preferably, the second active agent is a compound/substance that meets the requirements of Generally Recognized As Safe (GRAS) under sections 201(s) and 409 of the Federal Food, Drug, and Cosmetic Act. Preferably, such compounds/substances enhance the activity of the first active agent (e.g., CBD) in reducing alcohol consumption and/or treating alcohol hangover.

In some embodiments, the second active agent is an herbal ingredient such as an herbal extract or dried herb such as herbal powder. The herbal extract may be obtained by extraction using polar solvents such as water and alcohol or non-polar solvents. The herbal extract may be obtained by supercritical extraction, whereby the extract is made by extracting herbs with a gas, such as carbon dioxide, under a supercritical state, e.g., low temperature and high pressure. The herbal extract may also be an essential oil.

Major hangover signs may be due to toxicity, dehydration, and malabsorption of essential nutrients induced by alcohol and its metabolite acetaldehyde. Most of the alcohol that is consumed is metabolized to acetaldehyde by nicotinamide adenine dinucleotide-dependent alcohol dehydrogenase and further to acetic acid by nicotinamide adenine dinucleotide- dependent aldehyde dehydrogenase in the liver. Therefore, herbal ingredients with antioxidant activities can reduce oxidative stress and symptoms of hangover by mitigating plasma alcohol concentrations. Diverse antioxidative natural products including Nigella sativa, chamomile, green tea extract, vitamin C, and oriental medicinal preparations, have been used as candidates for alleviating the symptoms of alcohol hangover. Traditional plant medicines for hangover symptoms include root extracts of Pueraria lobata (McGregor, Alcohol, 2007, 41:469-78), Evodiae fructus extracts (Choi, et al., Biol Pharm Bull, 2006, 29:306-14), and a mixture of Korean medicinal herbs, i.e., Panax ginseng, Liriope platyphylla, andothers (Park, et al., Biol Pharm Bull, 2002, 25:1451-5).

The second active agent, as a dried herbal powder, an herbal extract, or an isolated or synthetic herbal compound, may be schizandra (Schizandra chinensis, chisandra berries), Zingiber officinale (e.g., ginger rhizome), Ginkgo biloba (e.g., ginkgo leaves), Glycyrrhiza glabra (e.g., licorice), Curcuma longa (e.g., turmeric rhizome), Gotu kola, red ginseng, prickly pear (e.g., prickly pear extract), chamomile, quercetin, dandelion root, milk thistle, vitamin C (sodium ascorbate), barley grass, green tea (e.g., green tea extract), Nigella sativa (black seed), herbal amino acids (glutamine, lysine, and cysteine), Cynara scolymus (globe artichoke), and combinations thereof. The second active agent may be an herbal mixture or an herbal mixture extract such as DTS20, an extract of the herbs: Viscum album L. (40%), Lycium chinense L. (30%), Inonotus obliquus (20%), and Acanthopanax senticosus H. (10%), wherein 1 gram of the dry extract contains sugars, polyphenols and other herb components. The dose for treating hangover can be 2.5 grams (Hong, Integr Med Res, 2016, 5:309-16; Yoon, et al., KSBB J, 2011, 26:433-8).

In some embodiments, the second active agent is Ginkgo biloba or an extract thereof, milk thistle or an extract thereof, or a combination thereof. Ginger and milk thistle as well as other herbal agents are helpful for reducing hangover. Silymarin, a standardized extract of milk thistle seeds, is known for protecting liver cells from being infiltrated by toxins. Silymarin is both an anti-inflammatory and antioxidant. Ginkgo biloba, or maidenhair, is a tree native to China that has been grown for thousands of years for a variety of uses. Gingko biloba seeds are also effective as an herbal remedy for hangover. Ginkgo biloba is rich in various medicinal properties and helps stimulate better digestion and metabolism. Consequently, Gingko biloba facilitates speedy removal of alcohol inside the brain and body to offer relief against a hangover.

In some embodiments, the second active agent is or contains one or more isolated or synthetic compounds that are known for reducing alcohol hangover symptoms. Such a compound may be an ingredient from an herbal extract.

Exemplary compounds include curcuminoids such as curcumin, alkaloids such as piperin, flavonoids such as quercetin, herbal amino acids such as glutamine, lysine, and cysteine, and antioxidants such as vitamin C and vitamin E.

The combination of the first active agent and the second active agent provides a greater effect in reducing alcohol consumption and alleviating hangover symptoms than either alone.

The second active agent can be formulated together with the first active agent in the same composition. Alternatively, the second active agent can be formulated in a separate composition, which can be the same or different from the design of the composition containing the first active agent. The second active agent can be administered before, after, or concurrently with the first active agent.

B. Vehicles

Anhydrous liposphere concentrate or ALC (also known as pro-nano liposphere or PNL), a type of self-emulsifying delivery system, enhances the oral bioavailability of poorly water soluble compounds by multiconcerted mechanisms which encompass enhanced solubility of the compounds (Elgart, et al., Pharm Res, 2013, 30:3029-44).

The carrier spontaneously forms nanodispersions containing nanoparticles (i.e., lipospheres) with an average diameter between 0.01 and 100 μm, preferably less than one micron, more preferably less than 500 nm, and most preferably less than 100 nm, upon addition to an aqueous medium. The carrier typically contains a lipid system, optionally a water-miscible solvent, and one or more surfactants and/or one or more phospholipids. The lipid from the lipid system forms the lipid core of the lipospheres, and the surfactants and/or phospholipids form the external coating of the lipospheres. The lipospheres can encapsulate the cannabinoid compound and other compounds from the active agents.

The ALC or PNL compositions may improve the bioavailability of the encapsulated compounds after oral intake and facilitate the delivery of these compounds to the enterocyte surface in their solubilized state. These compositions can aid overcoming the obstacles associated with oral administration of cannabinoid compounds because of (1) enhanced dispersibility in the aqueous milieu of the gastrointestinal (GI) tract; (2) increased intestinal surface area available for absorption due to the formation of nanoparticles; (3) decreased intestinal first pass metabolism (Phase I and Phase II) due to the formation of nanoparticles; and (4) enhanced lymphatic absorption which leads to bypass of the remaining metabolism occurring in the liver and a flatter absorption profile.

The compositions are dispersible concentrates that can generate dispersions when diluted in an aqueous medium. The carrier from the compositions is capable of spontaneously forming nanoparticles (i.e., lipospheres) upon addition to an aqueous medium. The lipospheres have a lipid core formed of one or more lipids from the lipid system of the carrier. They also have an external coating formed of one or more surfactants from the carrier. The outer coating may also contain one or more phospholipids from the carrier.

The lipospheres may have an average diameter of less than 500 nm, less than 200 nm, less than 100 nm, or less than 50 nm. Preferably, the lipospheres have an average diameter less than 100 nm.

The lipospheres may have a polydispersity index (Pdi) value of less than 0.8, less than 0.6, or less than 0.4. Preferably, the lipospheres have a Pdi value of less than 0.4.

The lipospheres can encapsulate the cannabinoid compounds or other hydrophobic compounds in their lipid core. The lipospheres may also encapsulate other compounds in the active agents separate from the hydrophobic core. CBD, CBN and other cannabinoid active agents are hydrophobic and can be dissolved in the ALC solution that contains a solid or liquid oil such as sesame oil, coconut oil, or palm oil that upon addition to water forms nanoparticles. Other hydrophobic herbal agents or actives can be dissolved in the ALC cannabinoid solution so when added to water forms nanodispersion that contain both types of active agents. If the added herbal active agent is insoluble in the cannabis ALC formulation, it can be added as solid dispersion or delivered independently before or after intake of the cannabis formulation. For the solid formulation where the formulation is absorbed in an absorbent such as porous silica or PEDIALYTE® powder, hydrophilic herbal agent can be mixed with the absorbent. The concentration of active agents in the ALC solution can be from about 1% to about 30% w/w where the concentration is dependent on the formulation ingredients and the desired particle size to be obtained upon addition to aqueous media. Optionally, the concentration of the first active agent (e.g., CBD) in the ALC solution is between about 0.5% and 15%, preferably between about 1% and 10%.

In some embodiments, the carrier contains a lipid system, a water-miscible solvent, one or more surfactants, and optionally one or more phospholipids.
1. Lipid System

The lipid system may contain one or more lipids such as monoglycerides, diglycerides, triglycerides, fatty acids, and combinations thereof. The lipids from the lipid system forms the lipid core of the lipospheres. In some embodiments, the lipid system contains triglycerides (TGs), including, but not limited to, long chain (C13-C21, such as C16-C20) TGs and/or medium chain (C6-C12, such as C8-C10) TGs. In some embodiments, the lipid system is or contains caprylic (C8) triglyceride, capric (C10) triglyceride, or a combination thereof.

In some embodiments, the lipid system is or contains plant oil, such as coconut oil, sesame oil, olive oil, peanut oil, lavender oil, castor oil, peppermint oil, orange oil, canola oil, palm oil, corn oil, or a combination thereof. In some embodiments, the lipid system is or contains refined or synthetic oil such as MIGLYOL® 812 (CAS: 37332-31-3; caprylic (C8)/capric (C10) triglyceride), MIGLYOL® 810 (caprylic (C8)/capric (C10) triglyceride, with a higher C10 content compared to MIGLYOL® 812), WITEPSOL® H grades such as WITEPSOL® H32 and WITEPSOL® H5 (WITEPSOL® H grades are triglycerides of saturated fatty acids C12-C18 with variable amounts of partial glycerides. They are hard fats with a low hydroxyl value of max. 15 (except for H19). They comprise mainly of triglycerides with portions of max. 15% of diglycerides and max. 1% of monoglycerides. WITEPSOL® H19 is a hard fat containing certain amounts of stabilized glyceryl ricinoleate which lead to a slightly higher hydroxyl value compared to the other H grades. They are characterized by a short gap between melting and solidification temperatures, showing a low tendency of posthardening (polymorphism). Shock cooling should be avoided because of their brittleness.), or combinations thereof. Preferably, the lipid system is or contains sesame oil. Preferably, the lipid system is or contains coconut oil.
2. Water-Miscible Solvent

The water-miscible solvent has low toxicity. It may have a water miscibility in the range of about 10% to full water miscibility at any weight or volume percentage of the formulation. In some embodiments, the water-miscible solvent is ethanol, isopropanol, N-methylpyrrolidone, ethyl lactate, ethyl acetate, DMSO, polyethylene glycol, glycerol, propylene glycol, or combinations thereof. Preferably, the water-miscible solvent is or contains ethanol. Preferably, the water-miscible solvent is or contains propylene glycol.
3. Surfactants

The surfactants form the external coating of the lipospheres, with the polar end pointing towards the outside of the lipospheres and the non-polar end pointing towards the inside of the lipospheres.

The surfactants may be anionic, cationic, nonionic, or zwitterionic compounds. In some embodiments, the surfactants are non-ionic surfactants, including, but not limited to, TWEEN® surfactants (polysorbates), such as TWEEN® 20, TWEEN® 65, and TWEEN® 80, SPAN® surfactants (sorbitan esters), such as SPAN® 80, BRIJ® surfactants (polyoxyethylene alkyl ethers), polyoxyethylene fatty acid ester surfactants such as CREMOPHOR® RH 40, sodium dodecyl sulfate; and combinations thereof. Preferably, the surfactants are or contain TWEEN® 20, SPAN® 80, and CREMOPHOR® RH 40.
4. Other Components

The carrier may further include one or more phospholipids, for example, egg or soy lecithin as well as phosphatidyl ethanolamine, phosphatidylic acid, phosphatidyl choline and synthetic phospholipids.

Alternatively, the carrier may further include one or more components containing phospholipid. In some embodiments, the phospholipid-containing component is lecithin.

C. Vehicles/Carriers—Fast-Dissolving Films

Fast-dissolving films (FDFs) can be used as an alternative oral delivery for pediatric and geriatric patients who experience difficulties swallowing traditional oral solid dosage forms. In some embodiments, the FDFs can dissolve within 10 minutes, preferably five minutes, more preferably two minutes, most preferably one minute in oral cavity after the contact with saliva. Compared to fast-dissolving tablets, the FDFs can be dissolved in the presence of less saliva, and thus do not require chewing or water for administration. Such films can deliver the active agents systemically through intra-gastric, sublingual or buccal route of administration and also can be used for local action.

The FDFs offer rapid absorption in the oral cavity for compounds/agents with low oral bioavailability and intensive first-pass metabolism. Longer release in the oral cavity can be achieved by making a slow dissolving film that sticks to the buccal and slowly dissolves over time. If buccal-tissue delivery is aimed, a two layer film is formed where a less permeable coat is applied.

The FDFs generally include one or more film-forming agents. The film-forming agents are typically water-soluble or water-dispersible. The film-forming agents can be small molecules with a molecular weight lower than 2,000 Da, polymers, or combinations thereof. Exemplary small molecules include mono-, di-, or oligo-saccharides such as lactose, glucose, and mannose, sugar alcohols such as mannitol, sorbitol, xylitol, erythritol, lactitol, and maltitol, and combinations thereof. Exemplary polymers include starch or modified starch such as corn starch, potato starch, and maize starch, hydroxypropylcellulose (HPC), hydroxypropyl methylcellulose (HPMC), polyvinylpyrrolidone (PVP), polyvinyl alcohol (PVA), polyalkylene glycols such as polyethylene glycol, poloxamers such as PLURONIC® F127, gelatin, alginates, carrageenin, dextran, maltodextran, natural gums such as acacia and xanthan gum, and combinations thereof. In some embodiments, the film-forming agent is HPC. In some embodiments, the film-forming agent is a mixture of HPC and PVP, or a mixture of HPC and poloxamer.

The FDFs can also include a surfactant, preferably a non-ionic surfactant such as those disclosed herein. In some embodiments, the surfactant is a TWEEN® surfactant (polysorbate), such as TWEEN® 20.

The FDFs can also include one or more excipients, such as flavorings (e.g., lemon oil), preservatives, etc.

The FDFs can be prepared by solvent casting that upon solvent evaporation a film is formed. Alternatively, the FDFs can be prepared via compression molding. In some embodiments, crosslinked acrylic acid (such as CARBOPOL®) can be added to the formulation to improve mucosal adhesion. In some embodiments, the first active agent and optionally the second active agent are directly loaded into the FDF such that they can be released and/or absorbed in the oral cavity. In other embodiment, the first active agent and optionally the second active agent are first loaded into an ALC as described herein, which is then loaded into the FDF.

D. Oral Dosage Formulations

Oral dosage formulations can be in liquid or solid form. The oral dosage formulations may include one or more pharmaceutically acceptable excipients. The oral dosage formulations may also include minerals such as magnesium, calcium, and/or potassium ions.

In some embodiments, the ALC compositions can be used as drops directly added to water, juice, or beverages such as soda before drinking. For example, the compositions may be placed in a dropping bottle. Alternatively, a bottle with a compartment loaded with any of the compositions can be used, where the compartment remains sealed and isolated from water or an aqueous medium, so that upon opening the bottle cap, the compartment is opened to release the composition which upon mixing with water or the aqueous medium forms an aqueous dispersion for drinking.

In some embodiments, the ALC compositions can be loaded in capsules such as gelatin capsules. Such formulations may release the compositions in the GI tract. Upon contact with gastric or intestinal fluids, such as stomach fluid, the compositions may spontaneously form nanoparticles (i.e., lipospheres) encapsulating compounds from the active agents.

In some embodiments, the oral dosage formulations may be or contain an aqueous dispersion of the ALC compositions for direct oral administration. The dispersion can be loaded in a sealed container.

In some embodiments, the ALC compositions may be absorbed on an absorbent, such as porous silica (such as NEUSILIN®), polymeric absorbent (such as POLY-PORE®), or any powdery material capable of absorbing large amount of the compositions, such as PEDIALYTE®. These semi-dry or dry formulations are dispersible solid lipid nanoparticles (DSLN) formulations. They can be loaded into capsules such as gelatin capsules or compressed into tablets, optionally together with one or more pharmaceutically acceptable excipients. Such formulations may release the compositions upon contact with aqueous media such as gastric or intestinal fluids in the GI tract. The released compositions may spontaneously form nanoparticles (i.e., lipospheres) that encapsulate compounds from the active agents.

In some embodiments, the ALC compositions can be added to an aqueous medium, which optionally contains cryoprotectant(s) and salts(s). The cryoprotectant can be selected from saccharides (such as mannitol, glucose, sucrose, and lactose) and polyalkylene glycols (such as polyethylene glycol). The resulting mixture is further lyophilized to form a solid formulation (such as a powder), wherein the solvents in the mixture (including water and the water-miscible solvent if present) are removed. The solid formulation can be loaded in capsules such as gelatin capsules or compressed into tablets, optionally together with one or more pharmaceutically acceptable excipients.

The FDFs can be prepared in the form of bite-sized pieces, and each piece may optionally corresponded to a single dose for direct consumption.

E. Kits

The compositions or formulations described herein can be packaged together with other components in any suitable combination as a kit useful for performing, or aiding in the performance of, the methods. It is useful if the components in a given kit are designed and adapted for use together in the methods.

The kits may contain, in one or more containers, any of the compositions or formulations. The kit may include instructions for use. The kit for ALC-based compositions or formulations may also provide an aqueous diluent such as, for example, water, saline, buffer, or buffered saline.

### III. Methods of Manufacturing

Solid oral dosage formulations of the ALC compositions, such as those formed by molded compression and loaded in gelatin capsules, can be made via various solidification methods, including spray drying, adsorption on solid materials, melt granulation, melt-extrusion, and lyophilization.

For example, the ALC compositions can be solidified by absorption onto an absorbent, e.g., porous silica particles, polymeric porous particles such as POLY-PORE®. This technique requires minimum time, equipment and cost; it also enables high content uniformity and high lipid exposure (such as about 70%).

Another route of solidification is lyophilization. In some embodiment, this technique may include dispersing the compositions in an aqueous medium to form nanoparticles (i.e., lipospheres), collecting the nanoparticles by ultracentrifugation, re-suspending the isolated nanoparticles in an aqueous medium optionally containing a cryoprotectant such as mannitol, and performing lyophilization.

Following solidification, a dry or semi-dry formulation can be obtained. The formulation can be filled in gelatin capsules or compressed into immediate or controlled release tablets, optionally together with one or more pharmaceutically acceptable excipients.

### IV. Methods of Use

Methods of controlling alcohol consumption and/or treating alcohol hangover using the compositions or formulations are also disclosed. The methods include orally administering to a subject in need thereof, an effective amount of any of the compositions or formulations to alleviate one or more symptoms of the hangover, such as headache, nausea, or dizziness. The compositions or formulations can be administered prior to, during, or after alcohol consumption.

In general, the amount of compositions or formulations that are required will vary depending on various factors such as the amount of alcohol consumed, the body weight of the subject in need thereof, and the time of administration.

An effective amount means the amount necessary to elicit an improved or desired response.

As will be appreciated by those of ordinary skill in the art, the effective amount of an agent may vary depending on such factors as the desired biological endpoint, the agent to be delivered, the nature of the site to which the agent is delivered, and the nature of the condition for which the agent is administered. For example, an effective amount of a composition or formulation for reducing alcohol hangover may be an amount sufficient to alleviate one or more symptoms of alcohol hangover, such as headache, tiredness, concentration problems, thirst, dizziness, nausea, cognitive impairment, and mood changes.

For example, it may be an amount sufficient to alleviate headache by 50% in intensity within about one hour, about 30 min, about 20 min, or about 10 min after administration of the composition or formulation.

A typical dose for cannabinoid compound (such as CBD) per treatment is in the range of 10 to about 100 mg, and the herbal composition to be used in combination of the cannabinoid compound can be from about 100 mg to about 3000 mg, depending on the herbal composition and extraction method.

### EXAMPLES

The present invention will be further understood by reference to the following non-limiting examples.

### Example 1

### Preparation and Characterization of CBD Compositions

Materials and Methods

The amphiphilic co-solvent and soy phospholipid were placed in a clean scintillation vial and heated to 45° C. until a homogenous solution was formed. Then the lipid designated to be the lipid core and surfactants were added. The mixture was gently stirred till a homogenous solution was formed. Further, CBD and/or herbal active agents were added, forming a non-aqueous composition, i.e., an anhydrous liposphere concentrate (ALC) composition or dispersible concentrate. Upon gentle agitation in aqueous phase, these compositions spontaneously formed drug-encapsulated nanodispersions.

Particle size and polydispersity index (Pdi) of the nano lipospheres present in the nanodispersions were determined using Zetasizer Nano ZS ZEN 3600 (Malvern Instruments Ltd, Malvern, UK). Prior to measurement, 100 μL of the ALC sample was vortex-mixed with 900 μL distilled water at 45° C. for 30 s to generate the nanodispersion containing lipospheres. The measurements on the water-diluted sample were performed using Capillary Cells (Malvern Instruments Ltd, Malvern, UK).

Results

The lipospheres formed in an optimized lipid-based composition, i.e., ES2-100-2, showed an average particle size of <100 nm and a Pdi value of <0.4. Table 1 demonstrates that eliminating one of the lipid components resulted in larger particles and increased dispersity. Additionally, it is evident that a specific ratio of surfactants is needed in order to achieve an optimal composition. Replacing TWEEN® 20 and SPAN® 80 with TWEEN® 65 that has the same hydrophile-lipophile balance as the combination of TWEEN® 20 and SPAN® 80 failed to yield the desired physical properties of the lipospheres (Table 2).

Solvent screening demonstrated that the tested solvents, including N-methyl-2-pyrrolidone (NMP), propylene glycol, ethanol, ethyl lactate, and isopropyl alcohol, resulted in compositions with different physical properties (Tables 1 and 3). Among them, ethanol, ethyl lactate, and isopropyl alcohol produced the optimal compositions for nano lipospheres.

TABLE 1
Compositions containing CBD. The measurements of Z-average size and Pdi (n =
3) were performed following 1:10 dilution of the compositions in water.

| | ES2-107-1 | ES2-107-2 | ES2-107-3 | ES2-107-4 | |
|---|---|---|---|---|---|
| | (without | (without | (without | (without | |
| | Span 80) | Tween 20) | Cremophor) | lecithin) | ES2-100-2 |
| Components | % w/w | % w/w | % w/w | % w/w | % w/w |
| Lecithin | 3.64 | 3.64 | 3.64 | 0 | 3.64 |
| Ethanol | 21.08 | 21.1 | 21.05 | 24.6 | 21.08 |
| CBD | 10 | 10 | 10 | 10 | 10 |
| Coconut oil | 10.88 | 10.86 | 10.89 | 10.87 | 10.9 |
| TWEEN ® 20 | 34.8 | 0 | 27.65 | 17.82 | 17.8 |
| SPAN ® 80 | 0 | 34.83 | 26.78 | 17.1 | 17 |
| CREMOPHOR ® RH 40 | 19.6 | 19.56 | 0 | 19.62 | 19.6 |
| Total | 100 | 100 | 100 | 100 | 100 |
| Visual inspection | Semisolid | Transparent | Transparent | Transparent | Transparent |
| of formulation | | liquid | liquid | liquid | liquid |
| d. (nm) | 324.8 | 439.6 | 670.0 | 127.8 | 26.62 |
| Pdi | 0.704 | 1.0 | 0.581 | 0.347 | 0.265 |

TABLE 2
Compositions containing different surfactants. The measurements of Z-average size and
Pdi (n = 3) were performed following 1:10 dilution of the compositions in water.

| | ES2-100-2 | ES2-107-6 | ES2-107-5 | ES2-107-1 | ES2-107-2 |
|---|---|---|---|---|---|
| Components | % w/w | % w/w | % w/w | % w/w | % w/w |
| Lecithin | 3.64 | 3.64 | 3.64 | 3.64 | 3.64 |
| Ethanol | 21.08 | 21.04 | 21.04 | 21.08 | 21.1 |
| CBD | 10 | 10 | 10 | 10 | 9.99 |
| Coconut oil | 10.88 | 10.84 | 10.84 | 10.88 | 10.87 |
| TWEEN ® 20 | 17.8 | 0 | 0 | 34.81 | 0 |
| SPAN ® 80 | 17 | 0 | 0 | 0 | 34.83 |
| TWEEN ® 65 | 0 | 0 | 34.77 | 0 | 0 |
| TWEEN ® 80 | 0 | 35.63 | 0 | 0 | 0 |
| CREMOPHOR ® RH 40 | 19.6 | 18.86 | 18.86 | 19.58 | 19.56 |
| Total | 100 | 100.01 | 100.01 | 99.99 | 99.99 |
| Visual inspection | Transparent | Transparent | Transparent | Transparent | Transparent |
| of formulation | liquid | liquid | liquid | liquid | liquid |
| Visual inspection | Clear | Clear | Cloudy | Milky-white | Milky-white |
| of dispersion | | | | | |
| d. (nm) | 26.62 | 29.86 | 91.81 | 324.8 | 439.6 |
| Pdi | 0.265 | 0.363 | 0.567 | 0.704 | 1 |

TABLE 3
Compositions with different water-miscible solvents. The
measurements of Z-average size and Pdi (n = 3) were
performed following 1:10 dilution of the compositions in water.

| | | ES2-108-2 | ES2-108-3 | ES2-108-4 |
|---|---|---|---|---|
| | ES2-108-1 | Isopropyl | Propylene | Ethyl |
| | NMP | alcohol | glycol | lactate |
| Components | % w/w | % w/w | % w/w | % w/w |
| Lecithin | 3.66 | 3.64 | 3.64 | 3.68 |
| Solvent | 20.98 | 21.06 | 21.03 | 21.09 |
| CBD | 10 | 10 | 10 | 10 |
| Coconut oil | 11.19 | 10.85 | 10.87 | 10.86 |
| TWEEN ® 20 | 17.64 | 18.05 | 17.82 | 18.03 |
| SPAN ® 80 | 17.08 | 16.93 | 17.02 | 17.09 |
| CREMOPHOR ® RH 40 | 19.46 | 19.48 | 19.64 | 19.25 |
| Total | 100 | 100 | 100.01 | 100 |
| Visual inspection | Transparent | Transparent | Transparent | Transparent |
| of formulation | liquid | liquid | liquid | liquid |
| Visual inspection | clear | clear | clear | clear |
| of dispersion | | | | |
| d. (nm) | 25.57 | 26.60 | 34.57 | 28.08 |
| Pdi | 0.315 | 0.165 | 0.390 | 0.134 |

Short, medium, and long chain triglycerides (TGs) or a combination of multiple TGs with different chain lengths were screened. Screening of long chain (C16-C20) TGs, such as olive oil, peanut, and sesame oil, demonstrated that sesame oil was the most suitable lipid system, resulting in an average particle size of 31.85 nm and a Pdi value of <0.4 (Tables 4 and 5). Using long chain (C13-C21) TGs, such as WITEPSOL®, resulted in solid or semi-solid non-aqueous compositions (Table 4). The compositions containing WITEPSOL® H32 (primarily containing saturated long-chain glycerides) or its combination (1:1) with sesame oil formed nano lipospheres with optimal particle sizes and Pdis (Table 4). The composition containing coconut oil and MIGLYOL® 812 also formed nano lipospheres with optimal physical properties (Table 5).

TABLE 4
Compositions containing different lipid systems. The measurements
of Z-average size and Pdi (n = 3) were performed following
1:10 dilution of the compositions in water.

| | ES2-100-2 | ES2-102-1 | ES2-102-2 | ES2-102-3 | ES2-102-4 |
|---|---|---|---|---|---|
| Components | % w/w | % w/w | % w/w | % w/w | % w/w |
| Lecithin | 3.64 | 3.64 | 3.66 | 3.64 | 3.64 |
| ethanol | 21.08 | 21.08 | 21.14 | 21.18 | 21.19 |
| CBD | 10 | 10 | 9.99 | 10 | 10 |
| Coconut oil | 10.9 | 0 | 0 | 0 | 0 |
| Sesame oil | 0 | 10.9 | 0 | 0 | 5.4 |
| WITEPSOL ® H32 | 0 | 0 | 10.9 | 0 | 5.44 |
| WITEPSOL ® H5 | 0 | 0 | 0 | 10.8 | 0 |
| TWEEN ® 20 | 17.8 | 17.88 | 17.8 | 17.8 | 17.59 |
| SPAN ® 80 | 17 | 16.94 | 17.04 | 17 | 17.15 |
| CREMOPHOR ® RH 40 | 19.6 | 19.58 | 19.5 | 19.56 | 19.59 |
| total | 100 | 100 | 100 | 100.02 | 100 |
| Visual inspection | Transparent | Transparent | Semisolid | solid | semisolid |
| of formulation | liquid | liquid | | | |
| d. (nm) | 26.62 | 31.85 | 22.31 | 27.60 | 28.25 |
| Pdi | 0.265 | 0.402 | 0.311 | 0.523 | 0.230 |

TABLE 5
Compositions containing different liquid systems. The measurements of Z-average size
and Pdi (n = 3) were performed following 1:10 dilution of the compositions in water.

| Components | | | | | | | | | | |
|---|---|---|---|---|---|---|---|---|---|---|
| (% w/w) | Lecithin | ethanol | CBD | oil | TWEEN ® 20 | SPAN ® 80 | CREMOPHOR ® RH 40 | total | d. (nm) | Pdi |
| ES2-106-8 | 3.64 | 21.08 | 10 | 10.88 | 17.8 | 17 | 19.6 | 100 | 24.91 | 0.316 |
| MIGLYOL ® 812 | | | | | | | | | | |
| ES2-106-1 | 3.65 | 21.1 | 9.98 | 10.88 | 17.79 | 17.03 | 19.56 | 100 | 51.28 | 0.839 |
| Olive oil | | | | | | | | | | |
| ES2-106-5 | 3.64 | 21.18 | 9.99 | 10.87 | 17.77 | 16.91 | 19.64 | 100 | 63.3 | 0.889 |
| Peanut oil | | | | | | | | | | |
| ES2-106-6 | 3.64 | 21.08 | 10 | 10.86 | 17.8 | 17 | 19.62 | 100 | 112.9 | 0.500 |
| Peppermint oil | | | | | | | | | | |
| ES2-106-2 | 3.61 | 21.17 | 9.99 | 10.98 | 17.7 | 16.96 | 19.59 | 100 | 202.28 | 0.416 |
| Lavender oil | | | | | | | | | | |
| ES2-106-7 | 3.66 | 21.04 | 10 | 10.84 | 18.04 | 16.88 | 19.56 | 100.02 | 217.6 | 0.279 |
| Castor oil | | | | | | | | | | |
| ES2-106-3 | 3.62 | 21.17 | 9.99 | 11.01 | 17.79 | 17.03 | 19.39 | 100 | 321.4 | 0.307 |
| Orange oil | | | | | | | | | | |
| ES2-106-4 | 3.63 | 21.06 | 9.98 | 10.98 | 17.74 | 17.19 | 19.52 | 100 | 465.6 | 0.469 |
| Canola oil | | | | | | | | | | |

### Example 2

### In Vivo Study of CBD Bioavailability

Materials and methods

Male Wistar rats, 275-300 g in weight, were used. All animals were deprived of food but not water 12 h prior to the conducted experiments. Animals were anesthetized for the period of surgery by intra-peritoneal injection of 1 mL/kg of ketamine-xylazine (9:1) solution. An indwelling cannula was placed in the right jugular vein of each animal for systemic blood sampling. The cannula was tunneled beneath the skin and exteriorized at the dorsal part of the neck. After completion of the surgical procedure, the animals were transferred to individual cages to recover overnight (12-18 h). During this recovery period, food, but not water, was deprived. Throughout the experiments, free access to food was available 4 h post oral administration. Animals were randomly assigned to the different experimental groups.

For bioavailability studies, nanodispersions were prepared 30 min before each experiment, by vortex-mixing of the CBD-containing ALCs with water (pre-heated to 37° C.) at a v/v ratio of 1:50 for 30 sec. The final CBD concentration in the nanodispersions was 3 mg/mL. The nanodispersions were administered to the animals by oral gavage (n=6-7). The administered dose of CBD was 15 mg/kg. The control group received CBD in a vehicle composed of propylene glycol:ethanol:water (4.5:4.5:1 v/v) at a concentration of 3 mg/mL (n=6).

Systemic blood samples (0.35 mL) were taken at 5 min pre-dose and 0.33, 0.66, 1, 1.5, 2, 4, and 6 h post-dose. To prevent dehydration, equal volumes of physiological solution were administered to the rats following each withdrawal of blood sample. Plasma was separated by centrifugation (4000 g, 7 min, 4° C.) and stored at −20° C. pending analysis.

Plasma aliquots of 150 μL were spiked with 20 μL of an internal standard, cannabigerol (CBG; 1 μg/mL). Acetonitrile (200 μL) was added to each test tube (tubes A) and vortex-mixed for 2 min. CBD and CBG were extracted by n-hexane (3 mL) that was added to each test-tube A, followed by 1 min vortex-mixing. After centrifugation at 4000 rpm for 10 min, the organic layer was transferred to fresh glass test tubes (tubes B) and placed under vacuum to remove the organic solvent. Then, tubes B were reconstituted with 80 mL of 20/80 (v/v) water/acetonitrile. The amounts of CBG and CBD were determined using a high-performance liquid chromatography (HPLC) system (Waters 2695 Separation Module) coupled with a mass-spectrometer (Waters Micro-mass ZQ, Waters Corporation, Milford, Mass.). Sample injection volume was 10 μL. The HPLC-MS conditions were as follows: an XTerra MS C18 column 3.5 μm 2.1×100 mm column (Waters, Milford, Mass.), an isocratic mobile phase of 20:80 (v/v) 2 mM ammonium acetate water solution:acetonitrile, a flow rate of 0.2 mL/min at 35° C. Retention time for CBD was 2.5 min. The detection masses (m/z) was 313.2 for CBD and 315.2 for CBG.

Results

In all studies, the CBD content in the formulation is 10% w/v. The ALC compositions of CBD showed 4-5 times higher AUC and Cmax, compared to the control sample containing non-formulated CBD.

### Example 3

### Solid Formulations of CBD Compositions

Materials and Methods

The adsorption-on-solid-carrier method was used to make solid formulations of CBD-loaded ALC. In a typical formulation, 0.1 to 1.0 mL of 10% CBD in ALC liquid was mixed in a dose of about 8 grams PEDIALYTE® solid powder composed of dextrose, citric acid, potassium and sodium citrate, zinc gluconate and flavors. Prior to use, the powder was loaded in PEDIALYTE® powder containers for mixing in water. Alternatively, ALC-absorbed PEDIALYTE® powder was loaded in hard gelatin capsules (1 gram containing 10 mg CBD) or compressed into 500 mg tablets containing 5 mg CBD. Higher amounts of CBD-loaded ALC in powder, capsules and tablets can be prepared by absorbing more concentrated CBD-loaded ALC. Commercially available synthetic or herbal extract CBD were used.

Another solidification technique was applied to obtain solid formulations. In this method, the CBD-loaded ALC solution was dispersed in a water solution containing the PEDIALYTE® composition or sugars such as dextrose or mannitol, and lyophilized. The amount of cannabinoids and herbal composition for treating hangover that were added to the aqueous medium was calculated based on the dose size to be used. Upon addition of ALC to the aqueous solution, nanoparticles were formed which after lyophilization, resulted in nanoparticles attached to the solid mixture formed due to water evaporation.

Results

In all studies, the CBD content in the formulation is 10% w/v. Lyophilization of the nanodispersions containing CBD-encapsulated nano lipospheres generated solidified formulations. The resulting powder was suspended in the same amount of water as that in the nanodispersions before lyophilization. The average size of the lipospheres in the resulting suspensions was slightly higher compared to the lipospheres in the original nanodispersions, but less than 500 nm.

Nanodispersions of blank lipid formulations without CBD were lyophilized overnight and then resuspended in the same amount of water as that in the nanodispersions before lyophilization. This process resulted in slightly larger particles. The optimal composition was ES2-102-2 with WITEPSOL® H32 as a lipid in the lipid system. This composition generated the smallest lipospheres after lyophilization, with an average size of 167.1 nm and a Pdi value of 0.28. The blank ALC is same as in Example 4 but the lipid is Tricaprin or Witepsol and the solvent is ethyl lactate.

### Example 4

### Pharmacokinetic Studies

Materials and methods

The tested composition was prepared as follows: soy lecithin was dissolved in ethanol and the other components, i.e., vegetable oil, CREMOPHOR® RH 40, TWEEN® 20, and SPAN® 80 were added. The mixture was gently stirred until a homogenous solution was formed. CBD (synthetic or plant extract) and herbal extract and internal standard ibuprofen were added, forming the CBD/ibuprofen pre-concentrate.

A typical blank composition without CBD is as follows:

| Component | % w/w |
|---|---|
| Lecithin | 4 |
| TWEEN ® 20 | 20 |
| SPAN ® 80 | 18 |
| CREMOPHOR ® RH 40 | 22 |
| Sesame oil | 12 |
| Ethanol | 24 |
| Total | 100 |

CBD (1 to 10% w/w) was dissolved in this solution.

Male Wistar rats (˜300 grams) were used to determine the CBD pharmacokinetics. The anhydrous liposphere concentrate was dispersed in water to form a nanodispersion of the CBD composition that was administered to the rats by oral gavage (n=3) with a dose of CBD at 15 mg/kg. CBD-mannitol lyophilized powder was administrated to rats via specially designed capsules, designated for oral administration. The required amount was given in two capsules, following a gavage with water in order to flush the content. Systemic blood samples (0.35 mL) were taken at 5 min pre-dose and 0.33, 0.66, 1, 1.5, 2, 4, 6, and 8 h post dose. To prevent dehydration, equal volumes of physiological solution were administered following each withdrawal of blood sample. Plasma was separated by centrifugation (4000 rpm, 10 min) and stored at −20° C. pending analysis.

Results

The CBD content in the CBD-mannitol lyophilized powder was determined by HPLC-UV analysis. It was found that on average 61 mg prepared powder contained 3.1 mg of CBD. This ratio was used for determining the amount of powder given to rats in order to obtain a 15 mg/kg dose.

Administration of CBD-ALC composition resulted in approximately 4-fold and 5-fold increase in AUC and Cₘₐₓ, respectively compared to the control group receiving the CBD-sugar capsule.

This experiment indicated that administration of CBD in ALC compositions resulted in a 4-fold increase in oral bioavailability of CBD, compared to the powder form. Administration of CBD in powder, led to low CBD concentration in the plasma and increased variability.

The difference between Example 2 and Example 4 is the negative control. Example 2 uses a CBD-containing liquid formulation as the negative control, whereas Example 4 uses CBD-sugar powder loaded in a capsule.

### Example 5

### Soft Drinks Containing CBD

Materials and Methods

PEDIALYTE® single dose anhydrous compositions containing 10 mg CBD and herbal additives for treating hangover were added to 100 mL water to form uniform clear dispersions containing lipospheres of particle size less than 100 nm. The dispersions were left at room temperature for one month and the stability of the dispersions was determined by visual evaluation of clarity and particle size.

Results

The dispersions remained stable after one month, the solution remain clear and the particles size was similar to the starting point.

### Example 6

### ALC Compositions Containing CBD and Herb Extracts

Materials and Methods

All chemicals, unless otherwise specified, were purchased from Sigma-Aldrich (Rehovot, Israel). CBD was purchased from THC Pharm GmbH, Germany. For the preparation of ALCs, sesame oil, coconut oil, TWEEN® 20, and SPAN® 80 were obtained from Holland Moran, Israel. The herbal compounds and Ginkgo leaves (Ginkgo biloba) and milk thistle (Silybum marianum) powder where purchased from Al Alim—Medicinal Herb Center, Hamovil, Israel.

Propylene glycol co-solvent and soy phospholipid were placed in a clean scintillation tube and heated to 45° C. until completely dissolved. Then sesame or coconut oil and TWEEN® 20 and SPAN® 80 were added, the mixture was gently stirred and heated to 45° C. until a homogenous solution was formed. Further, the active ingredients, CBD, Ginkgo biloba, and milk thistle were added. This non-aqueous formulation was gently stirred and heated to 45° C. until homogenous solution was formed. The dark clear formulation solution was loaded in different delivery systems including soft gelatin capsules and PEDIALYTE® powder.

Particle size and poly disparity index (Pdi) were determined using Zetasizer Nano ZS ZEN 3600 (Malvern Instruments Ltd, Malvern, UK). Prior to particle size and potential determination, 100 μL of the ACL formulation was vortex-mixed in 900 μL distilled water at 45° C. for 30 s, forming a dilution in a ratio of 1:10 (v/v). The measurements were taken using Capillary Cells (Malvern Instruments Ltd, Malvern, UK).

Results

The table below lists the components in an exemplary ALC composition containing sesame oil as the lipid system. The appearance of the composition was a clear dark solution, which remained stable (i.e., no change in color and no precipitation) for more than 30 days at room temperature. Stability is at room temperature, 25° C., stability is no change in appearance (clear light yellowish color) and particle size after dispersion in water at a 1:10 ratio, remain similar size.

| Component | % w/w |
|---|---|
| Lecithin | 3.5 |
| Propylene glycol | 20 |
| CBD | 1 |
| Gincago biloba | 5 |
| milk thistle | 5 |
| Sesame oil | 10 |
| TWEEN ® 20 | 18 |
| SPAN ® 80 | 18 |
| CREMOPHOR ® RH 40 | 18.5 |
| Total | 100 |

Upon addition of the oily formulation to an aqueous medium, the formulation spontaneously formed a drug-encapsulated nanodispersion.

The particle size after dispersion in water was analyzed in different time intervals to study the stability of the formulations. The particle size of the samples remained approximately 25 nm, during the studied time (i.e., 30 days). For all of the samples, the Pdi of less than 0.2 was obtained.

Similar compositions were prepared using coconut oil instead of sesame oil. The resulting products were clear and transparent, and remained stable at room temperature during the studies time. After dispersion of the formulations in water, an average particle size of <100 nm was observed.

The ACL compositions were further loaded into two exemplary delivery systems: (1) soft gelatin capsules and (2) PEDIALYTE® powder.

Packaging in soft gelatin capsules was performed as described below: 1 mL volume soft gelatin capsules were obtained from CAPSUGEL® or by emptying commercial soft gelatin capsules (originally loaded with oil) using a syringe and subsequently loading the ACL composition. The capsules were sealed with molten gelatin prepared by melting empty capsules.

Absorption onto PEDIALYTE® powder was performed as described below: spraying 1 mL ACL composition onto 5 grams of PEDIALYTE® powder while mixing to form a free flowing light brown powder that forms a clear solution upon adding water.

### Example 7

### Preparation of Fast-Dissolving Films

Materials and methods

Fast-dissolving films for the delivery of hangover remedies, CBD and active herbal agents, were prepared via solvent cast and evaporation to form uniform films.

Hydroxypropyl cellulose (HPC, Nippon Soda Co., Ltd.) with a viscosity of 151-400 mPa. (5% aqueous solution); PLURONIC® 127 was obtained from Sigma Aldrich, USA; TWEEN® 20 was obtained from Fisher Bioreagents; PLASDONE™ povidone was obtained from Ashland.

The firm-forming polymers were dissolved in ethanol along with the active agents and cast in silicon molds of 22.0 mm×25 mm. The molds were kept at 37° C. for the evaporation of ethanol to form the film.

Results

The weight ratio of each film was fixed to 150 mg/film. Size of the films was the size of the mold, 22.0 mm×25 mm. The thickness of the films was estimated to be between 100-150 μm. Table 6 provides exemplary formulations of the films.

TABLE 6
Appearance and solubility of fast-dissolving films. Each film is prepared
using 150 mg of the total material. Lemon oil was added at 1% for taste.
The percentages and ratios correspond to weight percentages and ratios.

| | | | % | | Gincago | | | |
|---|---|---|---|---|---|---|---|---|
| Film | | | Hydrophilic | TWEEN ® | biloba/milk | | | |
| No. | HPC | CBD | Polymer | 20 (%) | thistle (%) | Appearance | Oily | Solubility |
| 1 | 40% | 20% | 10 | 20% | 5/5 | Transparent | no | <1 min |
| | | | PLURONIC ® | | | | | |
| | | | F127 | | | | | |
| 2 | 40% | 20% | 20 | 20% | 10/0 | Transparent | no | <1 min |
| | | | PLURONIC ® | | | | | |
| | | | F127 | | | | | |
| 3 | 30% | 20% | 10 PVP | 20% | 0/10 | Semi- | no | <2 min |
| | | | | | | transparent | | |
| 4 | 30% | 20% | 20 PVP | 20% | 5/5 | Semi- | no | <1 min |
| | | | | | | transparent | | |

A small portion of Film No. 30 was tasted. The film's texture in mouth was very smooth. It did not stick to tongue but stuck well to the throat and dissolved very quickly (less than 30 sec in saliva). The taste of the films was tolerable.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

We claim:

1. A composition for treating alcohol hangover comprising:
a first active agent comprising at least one cannabinoid compound;
a second active agent effective in reducing one or more symptoms of alcohol hangover; and
a lipid carrier comprising a lipid, optionally a water-miscible solvent, and one or more surfactants,
wherein the lipid carrier spontaneously forms a liposphere dispersion upon addition to an aqueous medium, wherein the lipospheres have an average diameter of less than 500 nm,
wherein the lipospheres encapsulate the cannabinoid compound.

2. The composition of claim 1, wherein the first active agent is a cannabinoid extract from one or more cannabis plants, an isolated cannabinoid compound from a plant origin, or a synthesized cannabinoid compound.

3. The composition of claim 1, wherein the cannabinoid compound is selected from the group consisting of tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, cannabicitran, and derivatives thereof.

4. The composition of claim 3, wherein the cannabinoid compound is cannabidiol, tetrahydrocannabinol, or cannabinol.

5. The composition of claim 1, wherein the second active agent comprises an herbal ingredient.

6. The composition of claim 1, wherein the second active agent, as a dried herbal powder, an herbal extract, or an isolated or synthetic herbal compound, is schizandra, Zingiber officinale, Ginkgo biloba, Glycyrrhiza glabra, Curcuma longa, Gotu kola, red ginseng, prickly pear, chamomile, quercetin, dandelion root, milk thistle, vitamin C, barley grass, green tea, Nigella sativa, herbal amino acids, Cynara scolymus, and combinations thereof.

7. The composition of claim 6, wherein the second active agent is Ginkgo biloba, milk thistle, or a combination thereof.

8. The composition of claim 1, wherein the lipid carrier comprises one or more lipids selected from monoglycerides, diglycerides, triglycerides, fatty acids, and combinations thereof.

9. The composition of claim 8, wherein the lipid carrier comprises one or more triglycerides.

10. The composition of claim 1, where in the lipid carrier comprises an oil selected from the group consisting of coconut oil, sesame oil, olive oil, peanut oil, lavender oil, castor oil, peppermint oil, orange oil, canola oil, corn oil, MIGLYOL® 812, MIGLYOL® 810, WITEPSOL® H32, WITEPSOL® H5, and combinations thereof.

11. The composition of claim 10, wherein the lipid carrier comprises sesame oil.

12. The composition of claim 9, wherein the lipid carrier comprises caprylic triglyceride, capric triglyceride, or a combination thereof.

13. The composition of claim 1, wherein the water-miscible solvent is selected from the group consisting of ethanol, N-methylpyrrolidone, ethyl lactate, ethyl acetate, polyethylene glycol, glycerol, propylene glycol, and combinations thereof.

14. The composition of claim 13, wherein the water-miscible solvent is ethanol.

15. The composition of claim 1, wherein the surfactants are non-ionic surfactants, optionally selected from polysorbates, sorbitan esters, polyoxyethylene alkyl ethers, polyoxyethylene fatty acid ester surfactants, sodium dodecyl sulfate, and combinations thereof.

16. The composition of claim 15, wherein the surfactants comprises polysorbate 20, sorbitan oleate, and polyoxyl 40 hydrogenated castor oil.

17. The composition of claim 1, wherein the lipid carrier further comprises one or more phospholipids.

18. The composition of claim 17, wherein the lipid carrier comprises lecithin.

19. An oral dosage formulation of the composition of claim 1, comprising the composition and optionally one or more pharmaceutically acceptable excipients.

20. The formulation of claim 19, wherein the composition is loaded in gelatin capsules.

21. The formulation of claim 19, wherein the composition is dispersed in an aqueous medium, optionally further loaded in a sealed container.

22. The formulation of claim 19, wherein the composition is absorbed on an absorbent to form a semi-dry or dry formulation.

23. The formulation of claim 22, wherein the absorbent is selected from the group consisting of porous or mesoporous silica, polymeric absorbents, and PEDIALYTE® powder.

24. The formulation of claim 22, wherein the semi-dry or dry formulation is further loaded in a gelatin capsule or compressed in a tablet.

25. A lyophilisate of the composition of claim 1.

26. The lyophilisate of claim 25, wherein the composition is added to an aqueous medium and further lyophilized to form the lyophilisate.

27. The lyophilisate of claim 26, wherein the aqueous medium contains one or more cryoprotectants and/or one or more salts.

28. The lyophilisate of claim 27, wherein the cryoprotectants are selected from saccharides and polyalkylene glycols.

29. The lyophilisate of claim 25, wherein the lyophilisate is loaded in a gelatin capsule or compressed in a tablet.

30. The lyophilisate of claim 25, wherein the lyophilisate forms lipospheres in the gastrointestinal tract after oral administration.

31. A fast-dissolving film for treating alcohol hangover comprising:
a first active agent comprising at least one cannabinoid compound;
a second active agent effective in reducing one or more symptoms of alcohol hangover; and
one or more film-forming agents,
wherein the fast-dissolving film dissolves within 10 minutes, five minutes, two minutes, or one minute in oral cavity after contact with saliva.

32. The fast-dissolving film of claim 31, wherein the cannabinoid compound is cannabidiol, tetrahydrocannabinol, or cannabinol.

33. The fast-dissolving film of claim 31, wherein the second active agent comprises an herbal ingredient.

34. The fast-dissolving film of claim 31, wherein the one or more film-forming agents comprise a polymer, optionally selected from the group consisting of hydroxypropylcellulose, hydroxypropyl methylcellulose, polyvinylpyrrolidone, polyvinyl alcohol, alginates, polyalkylene glycols, poloxymaers, natural gums, and combinations thereof.

35. A method of treating alcohol hangover, comprising orally administering to a subject in need thereof, an effective amount of the composition, formulation, lyophilisate, or fast-dissolving film of claim 1, prior to, during, or after alcohol consumption.
